(19) United States
(12) Patent Application Publication
(10) Pub. No.: US 2023/0190820 A1
(43) Pub. Date: Jun. 22, 2023

(54) CELL HARVEST METHOD

(71) Applicants: The University of Melbourne, The University of Melbourne, Victoria (AU); Swinburne University of Technology, Hawthorn, Victoria (AU); University of Wollongong, Wollongong, New South Wales (AU); St. Vincent's Hospital (Melbourne) Limited, Fitzroy, Victoria (AU)

(72) Inventors: Peter F.M. Choong, The University or Melbourne (AU); Sam Lourdesan Francis, The University or Melbourne (AU); Serena Duchi, The University or Melbourne (AU); Carmine Onofrillo, The University or Melbourne (AU); Claudia Di Bella, Fitzroy (AU); Sanjeev Gambhir, Wollongong (AU); Simon Edward Moulton, Hawthorn (AU); Christopher Halkias, Hawthorn (AU); Cathal D. O'Connell, The University of Melbourne (AU); Nicholas Paul Reynolds, Hawthorn (AU); Gordon George Wallace, Wollongong (AU)

(21) Appl. No.: 17/927,415

(22) PCT Filed: May 27, 2021
(86) PCT No.: PCT/AU2021/050511

§ 371 Date: Nov. 23, 2022

(30) Foreign Application Priority Data
May 27, 2020 (AU) 2020901733

(51) Int. Cl. A61K 35/35; C12N 5/0775; A61K 41/00; A61K 47/42; A61K 47/18; A61K 47/36; A61P 19/02

(52) U.S. Cl. CPC A61K 35/35; A61K 41/00; A61K 47/36; A61K 47/42; A61K 47/183; A61P 19/02; C12N 5/0667; C12N 2509/00; C12N 2513/00; C12N 2533/54; C12N 2533/74; C12N 2537/10; C12N 2539/10

(57) ABSTRACT

The invention generally relates to cells and compositions comprising same for use in cell therapy, to methods of obtaining same, and to use of same in cell therapy. In one aspect, the invention provides a method for forming a cell composition from a tissue sample, the method comprising: providing a tissue sample comprising cells; contacting the sample with a polymer in binding conditions, said binding conditions being conditions that enable binding of cells in the sample to the polymer, so that said cells are bound to the polymer; culturing the cells bound to the polymer under conditions and for a time that allows the cell number to increase; providing conditions to induce a phase change of the polymer; thereby forming a cell composition from a tissue sample.

### FIELD OF THE INVENTION

The invention generally relates to cells and compositions comprising same for use in cell therapy, to methods of obtaining same, and to use of same in cell therapy.

### CROSS-REFERENCE TO RELATED APPLICATION

This patent application claims priority from Australian patent application no. 2020901733, the entire disclosure of which is hereby incorporated by reference.

### BACKGROUND OF THE INVENTION

Technologies to isolate, culture, expand, detach and deliver cells are of broad use in biomedicine, regenerative medicine, tissue engineering and stem cell therapy. Existing technologies require the sequential processing of cells on different materials, and typically with the addition of digesting enzymes and often animal derived enzymes which may irreversibly damage cells.

The isolation, culture, expansion, detachment and preparation of a cell population for implantation currently requires a series of steps which may each degrade the therapeutic capacity of the cells. Existing techniques to isolate a stem cell population (such as human adipose derived stem cells) from a tissue explant (such as the infrapatellar fat pad) rely upon the preferential attachment of a cell population onto cell culture plastics. Following removal of the undesired components of the explant, the desired population is then typically cultured on the same plastic substrate to expand the population to a useful number. When a large enough number of cells is reached, or when the cells have grown to confluency (a state where further expansion is constrained by the available surface area) the cells are detached from the cell culture plastic. The standard technique for detaching cells is to use digesting enzymes such as trypsin, collagenase or Dispase. The detached cell population is then typically mixed with another material (for example a hydrogel) for therapeutic delivery through injection or implant delivery, or to form a bio-ink for a subsequent biofabrication or 3D bioprinting step. In injection or extrusion-based procedures, the hydrogel material is often a shear-thinning material which protects the cells against shear stress induced damage.

This standard process contains a number of elements which can reduce the therapeutic capacity of the cell population:

Cell culture plastics have a mechanical stiffness several orders of magnitude above that of native tissues. Growth of stem cells upon such high-stiffness materials is known to reduce the stem-like phenotype of stem cells and/or induce senescence.

The enzymatic detachment processes typically require animal derived enzymes, which may be undesirable depending on the final use of the cells. These methods typically work through cleavage of cell surface proteins leading to dysregulation of cell function. Such methods can induce apoptosis in cells when exposed for longer time periods. Such methods unavoidably disrupt cell-cell interactions, which in many cases are desired (such as tissue spheroid or organoid cultures).

Finally, the detachment of cells from the tissue culture plastic and transfer to a biopolymer environment inevitably causes additional loss of cells through transfer errors.

Accordingly, there is a need for new and/or improved methods and compositions for preparing cells for implantation, and more generally, for improvements in procedures that utilise re-implantation of autologous cells.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the invention provides a method for forming a cell composition from a tissue sample, the method comprising:
providing a tissue sample comprising cells;
contacting the sample with a polymer in binding conditions, said binding conditions being conditions that enable binding of cells in the sample to the polymer, so that said cells are bound to the polymer;
culturing the cells bound to the polymer under conditions and for a time that allows, or causes, the cell number to increase;
providing conditions to induce a phase change of the polymer;
thereby forming a cell composition from a tissue sample.

In this aspect, the invention provides a method for forming a cell composition from a tissue sample, the method comprising:
providing a tissue sample comprising cells having chondrogenic potential;
contacting the sample with a polymer in binding conditions, said binding conditions being conditions that enable binding of cells in the sample to the polymer, so that said cells are bound to the polymer;
culturing the cells bound to the polymer under conditions and for a time that allows, or causes, the cell number to increase;
providing conditions to induce a phase change of the polymer;
thereby forming a cell composition from a tissue sample.

In this aspect, the invention provides a method for forming a cell composition from a tissue sample, the method comprising:
providing a tissue sample comprising cells having chondrogenic potential;
isolating the cells from the extracellular matrix in the tissue sample;
contacting the isolated cells with a polymer in binding conditions, said binding conditions being conditions that enable binding of the cells to the polymer, so that said cells are bound to the polymer;
culturing the cells bound to the polymer under conditions and for a time that allows, causes, the cell number to increase;
providing conditions to induce a phase change of the polymer;
thereby forming a cell composition from a tissue sample.

In this aspect, the invention provides a method for forming a cell composition from a tissue sample, the method comprising:
providing a tissue sample comprising cells having chondrogenic potential;
isolating the cells from the extracellular matrix in the tissue sample;
separating the isolated cells from substantially all the fat and/or liquid present in the tissue sample;
contacting the sample with a polymer in binding conditions, said binding conditions being conditions that enable binding of the cells to the polymer, so that said cells are bound to the polymer;
culturing the cells bound to the polymer under conditions and for a time that allows, or causes, the cell number to increase;
providing conditions to induce a phase change of the polymer;
thereby forming a cell composition from a tissue sample.

In another aspect, the present invention provides a method for treating an individual, the method comprising:
harvesting a tissue sample from an individual, or being provided with a harvested tissue sample from an individual;
contacting the sample with a polymer in binding conditions, said binding conditions being conditions that enable binding of cells in the sample to the polymer, so that said cells are bound to the polymer;
culturing the cells bound to the polymer under conditions and for a time that allows, or causes, the cell number to increase;
providing conditions to induce a phase change of the polymer, thereby forming a cell composition;
administering the cell composition to an individual;
thereby treating the individual.

In another aspect, the present invention provides a method for treating an articular cartilage defect in an individual, the method comprising:
harvesting a tissue sample from an individual, or being provided with a harvested tissue sample from an individual, said sample comprising cells having chondrogenic potential;
contacting the sample with a polymer in binding conditions, said binding conditions being conditions that enable binding of cells in the sample to the polymer, so that said cells are bound to the polymer;
culturing the cells bound to the polymer under conditions and for a time that allows, or causes, the cell number to increase;
providing conditions to induce a phase change of the polymer, thereby forming a cell composition;
administering the cell composition to an articular cartilage defect in the individual;
thereby treating the articular cartilage defect in an individual.

In this aspect, the present invention provides a method for treating an articular cartilage defect in an individual, the method comprising:
harvesting a tissue sample from an individual, or being provided with a harvested tissue sample from an individual, said sample comprising cells having chondrogenic potential;
isolating the cells from the extracellular matrix in the tissue sample;
contacting the isolated cells with a polymer in binding conditions, said binding conditions being conditions that enable binding of cells in the sample to the polymer, so that said cells are bound to the polymer;
culturing the cells bound to the polymer under conditions and for a time that allows, or causes, the cell number to increase;
providing conditions to induce a phase change of the polymer, thereby forming a cell composition;
administering the cell composition to an articular cartilage defect in the individual;
thereby treating the articular cartilage defect in an individual.

In this aspect, the present invention provides a method for treating an articular cartilage defect in an individual, the method comprising:
harvesting a tissue sample from an individual, or being provided with a harvested tissue sample from an individual, said sample comprising cells having chondrogenic potential;
isolating the cells from the extracellular matrix in the tissue sample;
separating the isolated cells from substantially all the fat and/or liquid present in the tissue sample;
contacting the sample with a polymer in binding conditions, said binding conditions being conditions that enable binding of cells in the sample to the polymer, so that said cells are bound to the polymer;
culturing the cells bound to the polymer under conditions and for a time that allows, or causes, the cell number to increase;
providing conditions to induce a phase change of the polymer, thereby forming a cell composition;
administering the cell composition to an articular cartilage defect in the individual;
thereby treating the articular cartilage defect in an individual.

In any aspect of the present invention, the cells remain bound to, or retained in or on, the phase changed polymer.

In another aspect, the present invention provides a cell composition, preferably a cell composition formed, obtained or obtainable by a method of the invention as described herein, preferably wherein the composition comprises cells having chondrogenic potential, preferably wherein the polymer comprises the following features: 1. cellular adhesion, 2. inducible phase change, preferably reversible phase change, and 3. crosslinkability. Preferably, the composition does not comprise fibroblasts.

In another aspect, the present invention provides a use of a cell composition formed by a method of the invention as described herein, or a cell composition of the invention as described herein, in the manufacture of a medicament for treatment of a condition requiring re-implantation of cells for said treatment.

In another aspect, the present invention provides a cell composition formed by a method of the invention described herein, or a cell composition of the invention as described herein, for use in the treatment of a condition requiring implantation of cells for said treatment.

In another aspect, the present invention provides a cell composition formed by a method of the invention as described herein, or a cell composition of the invention as described herein, when used for treatment of a condition requiring implantation of cells for said treatment.

In another aspect, the present invention provides a method of treatment comprising administering a cell composition formed by the method of the invention as described herein, or a cell composition of the invention as described herein, to an individual in whom said treatment is required.

In another aspect, the present invention provides a device or apparatus adapted for use in a method of the invention as described herein.

In another aspect, the present invention provides a kit for use, or when used, in a method of the invention, the kit comprising a polymer as described herein. Preferably, the kit further comprises written instructions to perform a method of the invention described herein.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Graphical representation of the steps involved in the repair concept. The the three features of the universal polymer are listed in the centre of the graphical abstract.

FIG. 2: (A) Graphical representation of the experimental design and associated timeframes of both control and rapid isolation groups. (B) Table demonstrating patient demographics of the three different patient lines tested. (C) Bar graphs representing the live cell count, cell viability, cell adhesion and hADSCs count post-selective adhesion for both control and rapid isolation groups. All measures were calculated using trypan blue staining. Data is presented as mean +/- standard error margin (SEM) between three biological replicates from three different patients (n=3), significant activity was calculated with unpaired t-test. Abbreviations: Infrapatellar fat pad (IFP) and human adipose-derived mesenchymal stem cells (hADSCs).

FIG. 3: (A) Representative phase contrast imaging analysis of hADSCs isolated off a Universal Polymer in the form of a layer, after 7 days. (B) The graph represents the metabolic activity of hADSCs expanded on an exemplary Universal Polymer (AlgRGD) in the form of a layer, in comparison with a control in which there were expanded on a standard plastic surface.

FIG. 4: Representative phase contrast and brightfield imaging analysis of hADSCs isolated off a Universal Polymer in the form of a layer and processed for the phase change. The graph represents the metabolic activity of hADSCs plated on plastic after the phase change. The Core/Shell image is a representative picture taken after the generation of the bioscaffold. The Core is the central compartment, while the Shell is the outer compartment surrounding the Core in a doughnut shape.

FIG. 5: Exemplary dimensions of 3D particles and layers and cell concentrations.

FIG. 6: Representative brightfield image of a AlgRGD 3D particle after cells attachment. 3D particles were created through the droplet/calcium chloride bath method. Expanded hADSCs (10,000 cells/mL) were seeded on 1% Alginate-RGD Particles in a Spinning Bioreactor. After 2 hours of interval spinning, cells were maintained in 6 well plate with 80 RPM spins.

FIG. 7: The graph represents the metabolic activity of hADSCs expanded on 3D particles in a spinner flask bioreactor.

FIG. 8: The graph represents the metabolic activity of hADSCs after treatment with the chelating agent.

FIG. 9: (A) Representative brightfield image of adherent cells present on a crosslinked AlgRGD 3D particle before phase change. (B) Representative brightfield image of the Bioink generated via phase change of AlgRGD 3D particles with 90 mM EDTA for 10 min.

FIG. 10: (A) Representative confocal images of a cross section of a Core/Shell Bioscaffold in which the compartments were labelled with two different fluorophores. The Core is the central compartment, while the Shell is the outer compartment surrounding the Core in a doughnut shape. (B) Representative confocal images of cross sections showing the accumulation of Collagen Type II under chondrogenic stimuli. The area selected with white borders highlight the Core compartment which is empty (black) at DAY1. The Core areas selected with white borders at DAY28 are full of Collagen Type II. (C) The graph shows gene expression level of the COL2A1 gene. (D) The graph shows the compressive modulus (10%-15%) of the Core/Shell Bioscaffolds.

FIG. 11: (A) Graphical representation of the experimental design. In brief, 2 full chondral defects were generated in the stifles of female sheep and treated, as indicated. A device (Biopen) was used to deliver a Core/Shell Bioscaffold. The repair Defect was analysed via Mechanical and Histological Analysis after 6 weeks from the surgery. (B) Representative images of Immunohistochemistry performed on the explants of the four study groups, as indicated. In the panels, the sections are shown with the cartilage layer facing the upper side. The Collagen Type I staining is physiologically detected in the bone compartment underneath the cartilage layer. The accumulation of unspecific collagen I in the cartilage compartment is evident in the BB and MF groups, while absent in the HH group. The Collagen type II (hyaline like cartilage) is physiologically present only in the cartilage layer. The images show significative accumulation of Collagen II-new cartilage only in the HH group. A modified O′Driscoll Score was applied to evaluate numerically the level of repair.

FIG. 12: Summary of the main results obtained in a 6 months large sheep study of a full chondral defect model. The 4 groups used in the study are: defect left empty (Empty); microfracture as gold standard treatment (MF); Core/Shell Bioscaffold treatment (Therapy); Core/Shell scaffold with no cells (Hydrogel). (A) 3D rendered reconstruction of MRI performed at 6 months-time point on sheep condyles: the images clearly show the superior extend of cartilage repair in the Therapy group (Core ALGRGD 1% + ADSC, Shell GelMA10% LAP0.1%) compared to the other control groups. The Empty defect also show evident signs of edema formation (black spot in the center of the defect). (B) The graph shows the International Cartilage Repair Society (ICRS) Score measured from the histological analysis at 6 months-time point for the 4 groups. (C) The graph shows the edema area measurement performed on in vivo MRI at 3 different time points (0, 3 and 6 months post-surgery) on sheep condyles for the indicated groups. This analysis clearly outline the presence of minimal inflammatory reaction after the Therapy treatment in comparison with the other 3 control groups.

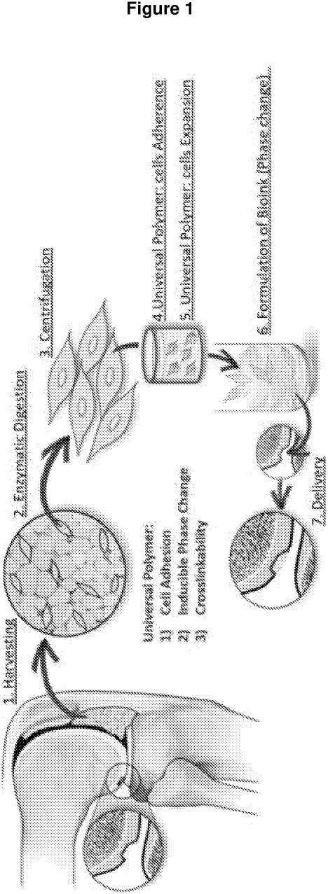

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

The present invention mitigates or entirely removes one or more of the problems in the prior art. A key aspect of the invention is the use of a single ‘universal’ hydrogel material for all of the steps of the process (isolation, purification, expansion, detachment and/or delivery).

The present invention relates to the use of biopolymer compositions which have capability to (i) isolate a desired cell population from a stromal mass by way of contact, (ii) provide a substrate for continued culture and/or proliferation of the desired population (while having a stiffness similar to that of native tissues) (iii) liquefy in a manner that causes encapsulation of cells within the material (obviating the need for harsh detachment treatments), (iv) subsequent delivery by means of injection or as a bio-ink formulation for 3D bioprinting.

An advantage of the present invention is that it involves the use of a single polymer composition as the biomaterial environment for isolation of cells, cell culture or expansion and surgical implantation. Specifically, the inventors have found that polymer substrates can be utilised to generate a clinically useful number of purified cells for (re)implantation. A further advantage of the process is that the extraction of cells from the harvested tissue directly into the substrate supports the viability of the cells prior to and after (re)implantation. This avoids the need for processing or formulation of cells post extraction and prior to (re)implantation.

Still further, the inventors have found that the re-implanted cells are functional. For example, with cells having chondrogenic potential, the method generates cells with the capacity to develop cartilage in damaged articular surfaces. Further, the substrate degrades after re-implantation, releasing the cells and enabling the cells to form new cartilage.

Some of the advantages arising from the method include:
Avoiding the need for animal derived enzymatic compounds such as trypsin;
Avoiding cell passaging prior to the therapy;
Limiting the malignant transformation risk;
Allowing cellular adaption to a substrate that will ultimately be used in re-implantation to support cells; and/or
Allowing for the generation of a closed system which can be automatically operated, sterile, controlled, customizable and/or deliverable.

The present invention avoids one or more elements of the prior art which can reduce the therapeutic capacity of the cell population, for example detaching and replanting cells for several passages with the use of enzymatic proteolytic agents.

The method, uses and compositions of the invention may find application in the provision of cells for implementation in cell therapy and/or surgical techniques. One particular example is in the provision of cells having chondrogenic potential to be used for repair or restoration of an articular surface. The method is now described further with reference to this specific implementation.

In a first step, the method comprises providing or having provided a tissue sample comprising cells. Generally, the sample is provided from the individual requiring treatment. It is a particular advantage of the method that it may be used in cell therapy and/or surgical techniques that are based on implementation of autologous cells.

A tissue sample may be obtained from tissue of the individual requiring treatment or may be taken from another individual. The tissue sample contains cells having the relevant function or the capacity to generate cells having the relevant function when (re)implanted into the individual. For example, the tissue sample contain cells with chondrogenic potential where the purposes is for use in producing cartilage (i.e. to treat a cartilage defect). In one embodiment, providing or having provided a tissue sample comprising cells does not involve a surgical step on a human or animal.

As used herein “chondrogenic potential” in the context of a cell means that the cell has the capacity to promote cartilage growth, particularly hyaline cartilage. This term is applied to cells which stimulate cartilage growth, such as chondrocytes, and to cells which themselves have the capacity to differentiate into a chondrocyte under appropriate conditions. Hyaline cartilage exists on the ventral ends of ribs, in the larynx, trachea, and bronchi, and on the articulating surfaces of bones.

A tissue sample that contains cells with chondrogenic potential may be sample of adipose tissue. Adipose tissue contains adult stem cells which may be mesenchymal stem cells, or related precursors, or cells derived from these cells that have chondrogenic, osteogenic and/or adipogenic potential. Accordingly, the present invention provides methods for treating defects that require cells of chondrogenic, osteogenic or adipogenic potential. In other words, “a tissue sample” or “a tissue sample comprising cells” may be a tissue sample that comprises cells having chondrogenic, osteogenic and/or adipogenic potential. For example, methods of the invention and cells or cell compositions produced therefrom could be used to treat bone defects, osteochondral defects, cartilage defects (not only articular cartilage), adipose tissue repair (e.g. breast reconstruction).

The mesenchymal stem cells, or related precursors, or cells derived from these cells have the capacity to form molecules of the extracellular matrix, and in particular molecules required for chondrogenesis and cartilage repair and restoration. Adipose derived stem cells (ADSCs) are particularly useful where the method is to be utilised in a procedure for cartilage repair or restoration. ADSCs may obtained from a number of different fatty tissues of the human or animal body. The ADSCs may be autologous or allogeneic.

In one particularly preferred embodiment, ADSCs are obtained from the infra patellar fat pad (IFP). The same tissue source (infrapatellar fat pad) can generate ADSCs that are known to display chondrogenic, osteogenic, and adipogenic potential. Given the 3 lineage differentiation potential, the cells could be used to treat bone defects, osteochondral defects, cartilage defects and adipose tissue repair.

An IFP may be obtained from an individual using standard techniques including those described herein. The IFP or sample therefrom may be harvested arthroscopically or upon open surgery. As described herein, an IFP generally comprises about 2 to 3 grams and about 8x10⁵ cells of which about 6x10⁵ cells are ADSC, therefore there are about 3x10⁵ ADSCs per gram of fat tissue in the IFP. Where a lesion has a greater volume, it may be necessary to utilise both or all fat pads, or to obtain ADSCs from other fat tissue. As described herein, the inventors have found that about 5 million ADSC per ml of polymer (e.g. hydrogel) is required to repair or restore a cartilage lesion. In one embodiment, the step(s) of harvesting IFP include any as described herein, including the Examples, particularly Examples 1 and 2.

In certain aspects of the invention, the method includes a step of isolating the cells from the extracellular matrix in the tissue sample. That isolation may be performed using one or more of mechanical disruption and enzymatic digestion, preferably both. For example, the IFP may be mechanically disrupted, minced or homogenized to isolate fat lobules. This can be achieved using a scalpel using standard techniques in sterile conditions within a few minutes. The purpose of the mechanical disruption is to improve exposure of the IFP to subsequent enzymatic digestion.

The disrupted IFP may then be subjected to collagenase digestion, the purpose of which is to separate the cells from extracellular matrix. Adipose tissue, including IFP generally contains a heterogenous mixture of cells, in particular including blood cells, adipocytes, fibroblasts and ADSCs. Generally the collagenase is used at a specific activity of about 2 U/ml. This enables the digestion time to provide separated cells to be reduced to 85 minutes or less, preferably 45 minutes or less, preferably 30 minutes or less. Despite the teaching in the art, the inventors have found that such digestion does not impact on the viability or potential of cells of the IFP for chondrogenesis. The digestion may be performed in conditions where the mechanically disrupted tissue is agitated. In one embodiment, the step(s) of mechanical and/or enzymatic digestion include any as described herein, including the Examples, particularly Examples 1 and 3.

At the completion of mechanical disruption and/or digestion, in certain aspects of the invention, the method includes separating the isolated cells from substantially all the fat and/or liquid present in the tissue sample. In this step the tissue sample may be the mechanically disrupted or enzymatically digested sample (or digest), and the sample or digest may be centrifuged to separate cells from a fat suspension and supernatant liquid. As defined herein the inventors have found that a cell pellet containing an appropriate number of cells for repair or restoration of an articular surface can be obtained in the cell pellet by centrifugation at 1000-2000 g for about 5-10 minutes, preferably 2000 g for 5 minutes. The centrifugation may be performed in the same vessel, i.e. tube, in which the mechanical disruption and/or enzymatic digestion occurred. In one embodiment, the step(s) of centrifugation include any as described herein, including the Examples, particularly Examples 1 and 3.

The cell pellet thus formed contains a heterogenous mixture of cells, including, as explained above, ADSCs and fibroblasts, and in addition, erythrocytes. Where the subsequent use of the cell composition formed by the method requires use of a composition that is devoid of erythrocytes s, the cell pellet may be resuspended in buffer for lysis of red blood cells, filtered to separate debris from viable cells and further centrifugation for about 400-800 g for about 2-5 minutes, 5 minutes at about 400 g to obtain a cell pellet. The pellet may then be resuspended in medium to enable the pellet to be further processed to purify desired cells and remove unwanted cells.

In all aspects of the present invention, the method includes the step of contacting the tissue sample, isolated cells, digest or cell pellet that has been resuspended in medium as the case may be with a polymer in binding conditions, said binding conditions being conditions that enable binding of cells in the sample, isolated cells, digest or cell pellet to the polymer, so that said cells are bound to the polymer. As mentioned, most tissue biopsies or samples, whether required for autologous re-implantation or otherwise, will tend to contain more than one cell type of interest. In some autologous uses it is particularly important to separate a first cell type from a second or further cell type existing in a sample before re-implantation of the cells in the individual requiring the relevant treatment. An aspect of the method enables separation of cells of different phenotype on the basis of preferential or selective binding to a polymer substrate. As exemplified herein the inventors have recognised that by contacting the cells of the tissue sample with a polymer under specific binding conditions it is possible to separate a first cell type from a second or further cell type i.e. to isolate a cell from a heterogeneous mixture of cells. Thus in one embodiment the method comprises the step of contacting the tissue sample, isolated cells or digest with a polymer in binding conditions, said binding conditions being conditions that enable the binding of cells to the polymer, and preferably to enable the binding of a first cell type to the polymer but not the binding of a second cell type to the polymer. In a subsequent method step the first cell type of interest may be separated from other unwanted cell types when the polymer having the first cell type bound thereto is separated from the 2nd, further or other cell types of the sample. In one embodiment, the step(s) of cell adherence to the polymer include any as described herein, including the Examples, particularly Examples 1, 4 and 7.

In one embodiment the step enables the binding of ADSCs to a polymer in conditions where other cells, and in particular, fibroblasts are unable to bind to the polymer. In this embodiment the polymer may be selected from the group consisting of gelatin, alginate or methacrylated derivatives thereof, ulvan and/or methacrylated derivatives thereof, or other polymer that comprises the following features: 1. cellular adhesion, 2. inducible phase change, preferably reversible phase change, 3. crosslinkability. The polymer may comprise a peptide or protein for cell adhesion. Typically, the peptide or protein binds to an extracellular matrix adhesion receptor, such as an integrin receptor. In one embodiment, the peptide or protein comprises an integrin binding motif, for example RGD. The peptide may comprise or consist of GGGGRGDSP, GRGDSP or GRGDS, or an amino acid sequence with 1 or 2 amino acid insertions, deletions, substitutions (preferably conservative substitutions) or a combination thereof, typically outside RGD. The polymer may be referred to as a biopolymer indicating suitability for in vivo use in a human or non-human animal.

In a particularly preferred embodiment, the polymer is in contact with (i.e. non covalently bound or attached to) a solid phase, such as a surface of a particle, vessel or device. In this embodiment the polymer may form a continuous or interrupted polymer surface on the solid phase, thus providing a surface for cells to bind to.

Particularly preferred particles, vessels or devices are those that are routinely used in cell culture. For example, a particle may be a bead or nanoparticle. A vessel may be a dish, flask, tube or other vessel used in, or for, cell culture.

Where the solid phase is a particle, the particle may be a gold particle and the polymer may be coated thereon.

A solid phase or particle may contain more than one type of polymer, one of which is used to bind to cells, one or more other polymers being used bind the polymer that has bound to cells to the solid phase or particle. Other polymers may be provided that have growth factors or factors for assisting in maintenance of stem cells. Thus a solid phase or particle may comprise a multilayered structure of polymers, or a blend of polymer that comprises at least one polymer capable of binding to cells under binding conditions.

Thus, in one embodiment, the polymer is capable of attaching to a solid phase of a particle, vessel or device, or capable of forming a particle in the binding conditions.

In other embodiments, the particle is formed from the polymer. For example, a particle may be comprised of, or consist of, alginate, collagen, gelatin and/or ulvan or a derivative thereof, or other polymer (including those described herein) that comprises the following features: 1. cellular adhesion, 2. inducible phase change, preferably reversible phase change, 3. crosslinkability.

Typically the polymer for use in the method may form a hydrogel at room temperature, may be liquefied by heating to a temperature above room temperature that does not impact on the viability or function of ADSCs, may be restored to a hydrogel on lowering of the temperature, and may be irreversibly cross linked, for example by visible light, UV radiation, enzymatic or electric field during or after re-implantation.

In any aspect or embodiment, the polymer is capable of reversible liquid-solid phase change. For example, the polymer may exist as a liquid, or semi-liquid, at room temperature and change to a solid, or semi-solid, by a change in temperature or in the presence of a chemical compound, for example a compound that can liberate divalent cations. Preferably the polymer has a reduction in flowability, e.g. has a phase change from a liquid to a solid, in the presence of an ionic crosslinking agent, for example a divalent cation such as Ca²⁺. Examples of suitable ionic crosslinking agents include calcium chloride (CaCl₂), calcium sulfate (CaSO₄) and calcium carbonate (CaCO₃). In another embodiment, the polymer is capable of a solid to liquid phase change caused by the chelation of a divalent cation, for example the divalent cation that caused a liquid to solid phase change. The chelation may occur by the presence of any chelating agent capable of chelating an ionic crosslinking agent, for example a divalent chelator such as ethylenediaminetetraacetic acid (EDTA), Ethylene glycol tetraacetic acid (EGTA), or citric acid.

The binding conditions may involve the contact of the cells of the sample with the polymer when the polymer is in a liquid state, a gel or a solid state.

An example of binding conditions that enable binding of cells in a sample to a polymer are as follows. The cell pellet that has been resuspended in medium may be cultured in, or on a vessel that contains one or more surfaces that have been coated with a polymer (such as GelMa (gelatin methacrylate)) for selective or preferential adherence of stem cells or ADSCs. The GelMa may be utilised at a concentration of about 10% w/w. The cells are maintained in this environment for about 30 minutes, a time period within which the inventors have found that stem cells or ADSCs may preferentially adhere to the polymer. The non-bound cells may be removed, for example by washing, thereby separating the polymer with attached stem cells or ADSCs from the sample to form a composition in the form of cells bound to the polymer.

Alternatively, the polymer may be alginate. Alginate is a naturally-occurring polysaccharide, obtainable from the cell walls of brown algae, that is composed of guluronic and mannuronic acid. Alginate has been shown to readily form hydrogels under mild conditions and Arg-Gly-Asp (RGD) integrin binding motifs have been added to improve cell adhesion. In any method of the invention, the polymer may be alginate-RGD in the form of 3D particles that can be created manually (using a needle/syringe combination), with a microfluidic system or via inkject 3D printing. The present inventors have generated alginate-RGD 1% particles and verified that cell adhesion and expansion is possible and the reversible chemical crosslinking feature does not affect cell viability. As discussed further below, alginate-RGD 3D particles were generated via crosslinking with 18-36 mM CaCl₂. An example of binding conditions that enable binding of cells in a sample to alginate-RGD 3D particles are as follows. Cells and Alginate-RGD 3D particles may be seeded into a bioreactor at an appropriate cell:sphere ratio (e.g. 10 cells to every particle). 3D particles and sphere are used herein interchangeably. The bioreactor may be filled with tissue culture medium (TCM). Spinning intervals involving short spinning periods, followed by longer non-spinning periods, may be undertaken to ensure cell adhesion to the particles.

In any aspect or embodiment, the polymer may comprise gelatin or a derivative thereof, for example gelatin methacryloyl (GelMA). In addition, or in the alternative, the polymer may comprise alginate or derivative thereof, for example sodium alginate.

In any embodiment, the polymer comprises alginate-RGD. Further, the polymer is capable of irreversible crosslinking. Therefore, the polymer is capable of reversible phase change, or reversible crosslinking, preferably mediated or caused by a chemical such as a divalent cation containing or liberating compound (i.e. ionic crosslinking), or by temperature changes, and is also capable of irreversible cross-linking, preferably mediated or caused by exposure to light (i.e. is photo-crosslinkable). Typically, photo-crosslinking is mediated by one or more reactive functionalities capable of photo-crosslinking such as methacryloyl, methacrylate, and methacrylamide groups in the polymer. In a preferred embodiment, the polymer comprises or consists of alginate, an RGD motif and a methacryloyl group.

In any aspect of the invention, the method includes a step of culturing the cells bound to the polymer under conditions and for a time that allows, or causes, an increase in cell number. Preferably, the conditions and time allows, or causes, at least 2 cycles of cell divisions, in other words allows a first division of the cells that initially adhere to the polymer, and then a division of the daughter cells from that first division. The conditions, such as tissue culture medium, will be known to the skilled person and relate to the specific cell type being expanded. There will be some variability in how quickly cell cultures expand and the number of cells on a random selection of polymer particles could be used to monitor the degree of expansion. However, after at least 5, 6 or 7 days in culture there should be at least 2 cycles of expansion of cells having chondrogenic potential. Therefore, an increase of about 3-4 times the original cell number should be present. Preferably, the culturing conditions and time allows an increase in number of stem cells, for example ADSCs. More preferably, the culture conditions allows an increase in cell number of stem cells and also priming of those stem cells to differentiate into a cell type of interest, for example, priming of ADSCs to form chondrocytes. Priming is performed on the same polymer without any passaging, thus continuing to avoid the use of any proteolytic agents such as trypsin. Accordingly, any method of the invention as described herein further includes a step of priming the cells at the same time or subsequently to culturing the cells that allows an increase in cell number.

In one embodiment, the step(s) of cell expansion on the polymer include any as described herein, including the Examples, particularly Examples 1, 5 and 8.

Bioreactor contents may be spun continually to allow for cell expansion whilst avoiding alginate sphere/disc agglomeration. In one embodiment, half of TCM volume is then removed and replaced with fresh TCM at an interval of 2-3 days. The protocol continues until the required amount of cells is reached.

The bioreactor is loaded with a ratio of cells and spheres, for example 10: 1,000,000 cells and 100,000 3D particles. All of these particles are suspended In TCM within the bioreactor. The bioreactor may then moved into an incubator and kept at 37° C., 5% CO2 - it is placed upon a Cimerac magnetic stirring apparatus at this time. The reactor impellor is then subjected to an interval protocol (this is controlled by the magnetic stirrer); the impellor may spin at 50 RPM for 2 minutes, after which a non-spinning interval period of 30 minutes is enforced. This cycle of stirring/non-stirring periods is continued for 4 hours, as these spin breaks are essential to allow for cell adhesion to the spheres. Once the 4 hour protocol is completed, the impellor is then reverted back to the standard stirring protocol of continual 50 RPM stirring without interval.

The bioreactor is then filled with extra TCM, to a final volume of 50 or 100 mL (this allows for appropriate cell culture conditions when the impellor is spinning). At a time point of 48 hours following the end of the stirring interval period, a total of 50% of the TCM within the bioreactor may be removed and replaced with fresh TCM - this is undertaken without removing spheres. The media continues to be replaced every 2-3 days following the previous TCM replacement, until the cell culture protocol is completed.

Alternatively, the cells may be cultured or expanded on any particle, vessel or device described herein.

In any aspect of the invention, the method includes a step of providing conditions to induce a phase change of the polymers. The phase change results in increase the flowability of the polymer. This can be achieved by heating the polymer (with the expanded cells still adhered) or by applying a chemical (e.g. EDTA) to reduce the degree of cross-linking within the polymer (with the expanded cells still adhered). Any treatment may, but typically does not, reduce the adherence of the cells for the polymer.

In one embodiment of this step, the step comprises heating the cell / polymer composition to melt the polymers, to increase the flowability of the polymers, or to liquefy the polymers. The purpose of this step is generally to liberate or to release the polymer/cell complex from a solid phase to which the polymer is bound and/or to enable the polymer/cell complex to be administered in a cell therapy or surgical procedure by utilising the properties of flow of the melted or liquefied composition, for example by extrusion, injection or 3D printing in the individual. In one exemplification of the method, the cells that remain bound to the polymer after the washing step described above may be subjected to heating by heating the vessel to which the GelMa is bound to about 37° C., the result of which is to melt the GelMa hydrogel thereby forming a composition having the desired properties of flow from which the solid phase to which the polymer was earlier attached can be removed. Thus, it will be recognised that polymer substrates for use in the method enable separation of cells on a solid substrate and release of cells from the solid substrate without affecting the viability of the desired cells.

Where the polymer forms a particle, the heating of the cell / polymer composition may liquefy the particle. In one embodiment, after the step in which a phase change is induced in the polymer, for example by heating, thereby liquefying the polymer, the cells that had bound to the polymer prior to the phase change induction remain bound to the polymer after the completion of the step. Thus, after a heating step, the composition does not become multiphasic, with for example, one phase containing polymer only and the other phase containing cells only. Instead, the cells remain bound to, or embedded in, the polymer after the heating step and this assists in the uniform delivery of the cells to a defect as the composition is administered during a re-implantation procedure.

In one particularly preferred embodiment the polymer has a melting temperature of below the temperature at which the desired functional properties of the cell of interest become compromised.

In a particularly preferred embodiment, the polymer selected for use in the method is one that is biocompatible with the individual and supports the cell in its delivery of the relevant cellular function when the cell is (re)implanted. This is advantageous as it enables the cell composition that has been heated to be utilised directly for re-implantation of the cells without further processing. As exemplified herein, the inventor has found that an alginate and/or gelatin derived polymer is particularly useful because it can be directly injected into an articular defect or lesion and subsequently degrades enabling the release of ADSC for migration to the articular surface and chondrogenesis.

In another embodiment, a chelating agent such as EDTA is applied to increase the flowability of the polymer, for example the alginate-RGD, and which does not substantially affect the viability of the desired cells. This particularly applies to an alginate-RGD polymer which has been reversibly crosslinked, or undergone a liquid to solid phase change, in the presence of an ionic crosslinking agent such as a divalent cation. The phase change also allows a step of mixing the cells with the liquefied polymer (e.g. alginate-RGD) to be performed. This mixture can then be administered in a cell therapy or surgical procedure, particularly to an articular cartilage defect. Alternatively, the mixture may be stored for later use, for example in cellular banking. In one embodiment, the EDTA is at a concentration of equal to, or less than, 250 mM, equal to, or less than, 200 nM, equal to, or less than, 150 nM, equal to, or less than 100 nM, or equal to, or less than, 90 nM. Preferably, the EDTA is applied for about 10 minutes.

In one embodiment, the step(s) of phase change of the polymers include any as described herein, including the Examples, particularly Examples 1, 6 and 9.

In certain aspects of the invention, the method includes a step of administering the cell composition to an articular cartilage defect in the individual. The cell composition may be the flowable polymer cell combination. Alternatively, the cell composition may be a mixture or emulsion of the cells and the flowable polymer.

The cell composition may be delivered to the site of (re)implantation arthroscopically (with ultrasound or imaging guidance) or upon open surgery. The delivered cell composition may be hardened by the activation of a photoinitiator. The photoinitiator may be activated with visible light. An example of a suitable photoinitiator is lithium phenyl-2,4,6 trimethylbenzoylphosphinate (LAP). The cell composition may be administered using a co-axial approach by which the cell composition forms a core around which a photocrosslinkable shell is applied. In any aspect or embodiment, a non-limiting example of a photocrosslinkable hydrogel comprises or contains a polymer comprising a reactive functionality capable of photo-crosslinking such as a methacryloyl group. For example, the hydrogel may comprise gelatin methacryloyl (GelMa) 10% (or 8% or 6%) and lithium phenyl-2,4,6 trimethylbenzoylphosphinate (LAP) 0.05% or 0.1%. This photocrosslinkable hydrogel may be cross-linked using conditions that are compatible with cell viability and chondrogenesis. Such conditions include 405 nm light source at 20 mW/cm² for 1 minute or 30 seconds.

In one embodiment, the step(s) of delivery include any as described herein, including the Examples, particularly Examples 1 and 10.

The present disclosure also includes the following according to the numbered clauses:

1. A method for forming a cell composition from a tissue sample, the method comprising:
providing a tissue sample comprising cells;
contacting the sample with a polymer in binding conditions, said binding conditions being conditions that enable binding of cells in the sample to the polymer, so that said cells are bound to the polymer;
culturing the cells bound to the polymer under conditions and for a time that allows the cell number to increase;
providing conditions to induce a phase change of the polymer, wherein the cells remain bound to the phased changed polymer;
thereby forming a cell composition from a tissue sample.

2. The method of clause 1, further comprising a step of isolating the cells from the extracellular matrix in the tissue sample.

3. The method of clause 2, wherein isolating the cells from the extracellular matrix is performed by mechanical disruption.

4. The method of clause 2, wherein isolating the cells from the extracellular matrix is performed by enzymatic digestion.

5. The method of any one of clauses 2 to 4, wherein isolating the cells from the extracellular matrix is performed by mechanical disruption and enzymatic digestion.

6. The method of any one of clauses 2 to 5, wherein isolating the cells separates the cells from any fat lobules in the sample.

7. The method of clause 4 or 5, wherein the enzymatic digestion is performed with collagenase.

8. The method of clause 7, wherein the collagenase is used at a specific activity of 2 U/ml for a period of 30 minutes or less.

9. The method of any one of clauses 2 to 8, further comprising the step of separating the isolated cells from substantially all the fat and/or liquid present in the sample.

10. The method of clause 9, wherein separating the isolated cells may be performed by centrifugation.

11. The method of clause 10, wherein the centrifugation is performed at about 2000 g for about 5 minutes to form a cell pellet.

12. The method of clause 11, wherein the cell pellet is resuspended in a buffer for lysis of red blood cells.

13. The method of clause 12, further comprising filtering the cells in the lysis buffer to separate debris from viable cells and further centrifugation for about 5 minutes at about 400 g to obtain a further cell pellet.

14. The method of clause 1, wherein the polymer is capable of attaching to a solid phase of a particle, vessel or device, or capable of forming a particle, in said binding conditions.

15. The method of any one of clauses 1 to 14, wherein the polymer is capable of binding to cells in said binding conditions that are human adipose derived stem cells (ADSCs) or hADSC precursor cells, or to cells that are derived from hADSC that are chondrogenic or that have chondrogenic potential.

16. The method of any one of clauses 1 to 15, wherein the polymer is not capable of binding to fibroblasts in said binding conditions.

17. The method of any one of the clauses 1 to 16, wherein the polymer comprises a peptide or protein.

18. The method of clause 17, wherein the peptide or protein binds to an extracellular matrix adhesion receptor.

19. The method of clause 18, wherein the extracellular matrix adhesion receptor is an integrin receptor.

20. The method of clause 17, wherein the peptide or protein comprises an integrin binding motif.

21. The method of clause 20, wherein the integrin binding motif is RGD.

22. The method of clause 17, wherein the peptide comprises or consists of GGGGRGDSP (G4RGDSP), G4RGDSY, GRGDSP or GRGDS, or an amino acid sequence with 1 or 2 amino acid insertions, deletions, substitutions (preferably conservative substitutions) or a combination thereof.

23. The method of any one of clauses 1 to 22, wherein the polymer is capable of reversible liquid-solid phase change.

24. The method of any one of clauses 1 to 23, wherein the polymer is capable of a liquid to solid phase change caused by an ionic crosslinking agent.

25. The method of clause 24, wherein the ionic crosslinking agent is a divalent cation.

26. The method of clause 25, wherein divalent cation is Ca²⁺.

27. The method of any one of clauses 1 to 26, wherein the polymer is capable of a solid to liquid phase change caused by the chelation of an ionic crosslinking agent.

28. The method of clause 27, wherein the chelation occurs by the presence of a chelating agent capable of chelating an ionic crosslinking agent.

29. The method of clause 28, wherein the chelating agent is EDTA.

30. The method of any one of the clauses 1 to 29, wherein the polymer comprises gelatin or a derivative thereof.

31. The method of clause 30, wherein the gelatin polymer is gelatin methacryloyl (GeIMA).

32. The method of any one of clauses 1 to 31, wherein the polymer comprises alginate or derivative thereof.

33. The method according to clause 32, wherein the polymer comprises alginate-RGD.

34. The method according to any one of clauses 1 to 33, wherein the polymer is capable of photo-crosslinking.

35. The method according to clause 34, wherein the photo-crosslinking is mediated by a reactive functionality capable of photo-crosslinking present in the polymer.

36. The method according to clause 35, wherein the reactive functionality is a methacryloyl group.

37. The method of any one of clauses 1 to 36, wherein the polymer comprises alginate, an RGD motif and a methacryloyl group.

38. The method of any one of clauses 1 to 37, wherein the tissue sample comprises a first cell type and a second cell type and wherein the binding conditions enable the binding of the first cell type to the polymer and wherein the binding conditions do not enable binding of the second cell type to the polymer.

39. The method of clause 38, wherein separation of the polymer from the tissue sample forms a cell composition consisting of cells of the first cell type, and forms a waste stream comprising cells of the second cell type.

40. The method of clauses 38 or 39, wherein the first cell type is a hADSC or chondrogenic cell and the second cell type is a fibroblast.

41. The method of any one of clauses 1 to 40, wherein the tissue sample is obtained from the infrapatellar fat pad.

42. The method of clause 41, wherein the fat pad has a weight of about 2 to 3 g.

43. The method of any one of clauses 1 to 42, wherein the polymer is in the form of a 3D particle.

44. The method of clause 43, wherein the sample is contacted with the 3D particle in a bioreactor at a cell:particle ratio of about 10 cells to every particle.

45. The method of any one of clauses 1 to 44, wherein the step of culturing the cells allows at least 2 cycles of cell divisions.

46. The method of any one of clauses 1 to 44, wherein the step of culturing the cells is for a period of at least 5, at least 6 or at least 7 days.

47. The method of any one of clauses 1 to 44, wherein the step of culturing the cells results in an increase of about 3-4 times the original cell number.

48. The method of any one of clauses 1 to 44, wherein the step of culturing the cells results in about 5 million cells.

49. The method of any one of clauses 1 to 48, wherein when the tissue sample contains stem cells, preferably ADSCs, the method further comprises the step of priming of those stem cells to differentiate into a cell type of interest, for example, priming of ADSCs to form chondrocytes.

50. The method of clause 49, wherein the priming step occurs at the same time or subsequent to culturing the cells that allows an increase in cell number.

51. The method of any one of clauses 1 to 50, wherein the conditions to induce a phase change of the polymer is heating.

52. The method of any one of clauses 1 to 51, wherein the conditions to induce a phase change of the polymer is application of a chelating agent.

53. The method of clause 52, wherein the chelating agent is EDTA.

54. The method of any one of clauses 51 to 50, wherein the phase change increases the flowability of the polymer enabling the cell composition to be administered to an individual at room temperature by injection, extrusion or 3D printing.

55. The method of clause 51, wherein the heating step comprises heating the cell composition to a temperate that does not affect the viability of the cells in the cell composition.

56. The method of any one of clauses 1 to 55, wherein the polymer has a melting temperature of about 25 to about 30° C.

57. A method for treating an individual comprising:
forming a cell composition according to any one of the preceding clauses, or being provided with a cell composition formed according to any one of the preceding clauses;
administering the cell composition to the individual,
thereby treating the individual.

58. The method of clause 57, wherein the cell composition is formed from a tissue sample obtained from the individual.

59. The method of clause 58, wherein the cell composition is formed from a tissue sample obtained from an infrapatellar fat pad of the individual.

60. The method of any one of clauses 57 to 59, wherein the cell composition is administered by injection, extrusion or 3D printing.

61. The method of any one of clauses 57 to 60, wherein the cell composition is administered with a further polymeric composition so that the further polymeric composition coats the cell composition as the cell composition is administered to the individual.

62. The method of clause 61, wherein the further polymeric composition is photocrosslinkable.

63. The method of any one of clauses 57 to 62, wherein the individual has a condition of an articular surface requiring repair or restoration.

64. The method according to any one of clauses 57 to 63, wherein the cell composition is administered to an articular surface requiring repair or restoration.

65. The method according to any one of clauses 57 to 64, wherein the cell composition is administered arthroscopically, preferably with ultrasound or imaging guidance.

66. The method according to any one of clauses 57 to 64, wherein the cell composition is administered upon open surgery.

67. The method according to any one of clauses 57 to 66, wherein the delivered cell composition is hardened by the activation of a photoinitiator.

68. The method of clause 67, wherein the photoinitiator is activated with visible light.

69. The method of clause 68, wherein the photoinitiator is LAP.

70. The method of clause 68 or 69, wherein a 405 nm light source at 20 mW/cm² is applied for 1 minute or 30 seconds.

71. A cell composition obtained by a method of any one of clauses 1 to 56.

72. A cell composition obtainable by a method of any one of clauses 1 to 56.

73. Use of a cell composition of clause 71 in the manufacture of a medicament for treatment of a condition requiring re-implantation of cells for said treatment.

74. A cell composition of clause 71 for use in the treatment of a condition requiring implantation of cells for said treatment.

75. A cell composition of clause 71 when used for treatment of a condition requiring implantation of cells for said treatment.

76. A kit for use, or when used, in a method of any one of clauses 1 to 70, the kit comprising a polymer as defined in any one of clauses 1 to 70.

77. The kit of clause 76, further comprising written instructions to perform a method of any one of clauses 1 to 70.

78. Use of a cell composition of clause 72 in the manufacture of a medicament for treatment of a condition requiring re-implantation of cells for said treatment.

### EXAMPLES

### Example 1 - Materials and Methods

### Harvesting

Human IFP was opportunistically harvested from three patients undergoing elective total knee arthroplasty with informed consent after ethics approval [HREC/16/SVHM/186]. Harvested IFPs tissues were washed with PBS 1X (Sigma-Aldrich, St. Louis, MO, USA) under a biosafety hood to remove blood. Next, mechanical breakdown was achieved with a scalpel to isolate fat lobules, and the tissue was then weighed to perform the next phases of the isolation.

### Enzymatic Digestion and Centrifugation

### Standard

Briefly, chemical digestion was achieved with 1 mg/mL collagenase type 1 (Worthington Biochemical, Lakewood, NJ, USA) for 3 hours at 37° C. under constant agitation on a shaker at 250 rpm. The sample was centrifuged at 2100 g for 10 minutes, and the resulting cell pellet was resuspended in PBS 1X before being filtered through a 100 µl nylon cell strainer (Millipore, Darmstadt, Germany) and centrifuged at 400 g for 5 minutes. The remaining pellet was then resuspended in 5 ml of Red Cell Lysis Buffer (160 mM NH₄Cl; Sigma Aldrich) for 10 minutes and filtered through a 40 µl nylon cell strainer (Millipore, Darmstadt, Germany). The sample was centrifuged at 400 g for 5 minutes, the cell pellet was resuspended in 500 µl of complete culture media [low glucose DMEM (St. Louis, LA, USA) supplemented with 10% Foetal bovine serum FBS (Gibco, Thermo Fisher Scientific Inc, Waltham, MA, USA), 100 U ml⁻¹ Penicillin and 100 µl ml⁻¹ Streptomycin solution (Gibco), 2 mM L-Glutamine (Gibco), and 15 mM HEPES (Gibco), 20 ng ml⁻¹ epidermal growth factor and 1 ng ml⁻¹ fibroblast growth factor (R&D Systems Inc., Minneapolis, MN, USA)]. Cell count and viability were calculated using a haemocytometer and trypan blue based live-dead staining. Cells were evenly plated on non-coated plastic 6-wells plates and incubated for 24 hours allowing for cellular adherence. Non-attached material was discarded, and the adherent cells were washed once with PBS 1X before detaching with 500 µl of trypsin, followed by incubation for 3 minutes allowing for cellular de-attachment. Next, 1 ml of complete hADSCs culture media was added to each well to neutralise the trypsin. Samples were then centrifuged at 400 g for 5 minutes, the resulting pellet was resuspended in 500 µl of complete hADSCs culture media. The adherence percentage and cell count were calculated using a haemocytometer and trypan blue based live-dead staining under light microscopy. Cells were replated at a concentration of 5000 cells/cm² onto non-coated plastic flasks and expanded for down line investigations.

### Rapid

All steps were identical to the control isolation protocol apart from the following two changes: i) Chemical digestion was achieved in 30 minutes with 1 mg/mL collagenase (Worthington Biochemical, Lakewood, NJ, USA), ii) Cells were plated on the Matrigel-coated plastic 6-wells plates (Lifesciences, Corning, Tewksbury, MA, USA), and incubated for 30 minutes to allow for cellular adherence. Wells were coated as per the manufacturers’ protocol.

### Cells Adherence on Layer

1 mL of Alginate1% RGD (NOVATECH, Norway; the RGD is GRGDSP) was casted on a well of a 6 well-plate and left at -20C for 30 minutes to generate a flat surface. The Alg-RGD was then crosslinked in a reversible way by adding 600ul of CaCl2 180 mM and left in contact for 30 minutes at Room Temperature. The excess of CaCl2 was then removed and the coated well rinse with complete cell culture media for 2 times. Cells isolated from IFP with the rapid approach (see Examples 2-3) were seeded on a Universal Polymer layer and growth in a CO₂ cell incubator with 0.5 mL of cell culture media, and morphology was observed during time with a Evos microscope.

### Cells Expansion on Layer

The expansion of hADSCs on a Universal Polymer on a form of a layer with the cell titer metabolic assay (Promega) following the manufacturer’s instructions.

### Formulation of Bioink (Phase Change on Layer)

To induce the phase change of hASDSCs expanded on a Universal Polymer (AlgRGD1%) in a form of a layer, 0.3 mL of EDTA 250 mM diluted in PBS were added on top of the layer. The phase change was performed in the cell culture incubator for 10 minutes. The solution containing the complete liquified Alg-RGD and the cells, was then divided in two aliquots.1) 0.75 mL were spun at 1500 rpm 3 min and cells pellet resuspended in 1 mL and plated on a well of a 6well plate for the metabolic activity test at day 1 and 4 after plating. Cell titer metabolic assay (Promega) was used following the manufacturer’s instructions. 2) 0.75 mL were loaded onto the Core compartment of a co-axial device (Biopen). The Shell compartment was constituted of GelMa 8% and the LAP photoinitiator at 0.1% concentration. The extrusion was performed in a Core/Shell ratio of 60:40 at speed 6ul/sec inside a PDMS mold of 80ul volume. The hardening of the Shell compartment was achieved via photo-crosslinking at 400 nm wavelength LED light, at 20 mW/cm2 for 60 sec. The bioscaffolds were then removed from the PDMS mold, whased in PBS and transferred to a 24 well plate for chondrogenesis experiment.

### Cells Adherence on 3D Particles

Alginate spheres are produced by dropping a 1% (w/v) alginate solution (also contains the amino acid-based molecule RGD) onto a bath of (18 - 180 mM) CaCl2. Following the creation of alginate spheres hADSCs are allowed to adhere to spheres. The attachment process involves placing hADSCs and 3D particles into a BioReactor spinner flask chamber in 1:1 to 20:1 ratio. The next step involves allowing the BioReactor spinner flask impellor to spin at 50 RPM in intermittent time intervals (2 minutes of spinning, follow by a 30 min rest period where no spinning occurs), which encourages interaction between spheres and hADSCs, while also allowing hADSCs time to adhere to spheres. This entire process was conducted in 10 ml of media.

### Cells Expansion on 3D Particles

Once hADSCs are attached, the amount of media present in the BioReactor spinner flask was increased to 25 ml and a protocol of continual impellor spinning at 50 RPM was then undertaken for 7 days, with the BioReactor spinner flask left to incubate at 37° C. and 5% CO₂.

50% of the cell media present in the spinner flask was removed and replaced every 2-3 days, without removing any spheres from the inner flask environment.

The expansion of hADSCs on a Universal Polymer in the form of a 3D particle was assessed with the Presto Blue metabolic assay (Thermo Scientific) following the manufacturer’s instructions.

### Formulation of Bioink (Phase Change on 3D Particles)

The media within the Bioreactor spinner flask chamber was removed, leaving only the populated spheres within. The AlgRGD particles were then dissolved by a 10 minute exposure to a solution containing a biologically-relevant media substitute (e.g. calcium-free PBS) and 90 mM EDTA at 37° C. and 5% CO₂.

### Bioink Formulation

Gelatin methacryloyl (GelMa) was synthesized by TRICEP (https://www.tricep.com.au/). Briefly, the material was dissolved to a final concentration of 100 mg ml⁻¹ GelMa in sterile PBS (Sigma-Aldrich), containing 100 U ml⁻¹ penicillin and 100 µg ml⁻¹ of streptomycin (Gibco). Porcine Gelatin was provided by Sigma and used at 80 mg ml⁻¹ in sterile PBS containing 100 U ml⁻¹ penicillin and 100 µg ml⁻¹ of streptomycin.

### Delivery

Co-axial extrusion was performed using the handheld extrusion system (Biopen). Briefly, both Biopen chambers were loaded with:

SHELL: 10% GelMa and 0.1% w/v Lithium-acylphosphinate (LAP) (Tokyo Chemical Industry Co., Tokyo, Japan).

CORE: 8% Gelatin mixed with 10 × 10⁶ cells ml⁻¹hADSCs.

Samples were extruded with the Biopen into PDMS moulds to produce disc-like shaped bioscaffolds (height =2 mm, diameter =7 mm). Immediately after extrusion the samples were then UV irradiated at room temperature for 60 seconds, using a 405 nm UV source with a light intensity of 20 mW/cm². The generated bioscaffolds were then transferred to a 24 well plastic plate, washed three times in PBS 1X and 1 mL of chondrogenic or control medium was added to each well. The control medium consists of DMEM high-glucose (Lonza), 100 U ml⁻¹ penicillin and 100 µg ml⁻¹ of streptomycin (Gibco), 1X Glutamax (Gibco), and 15 mM HEPES (Gibco), while the chondrogenic medium consists of DMEM high-glucose (Lonza), 100 U ml⁻¹ penicillin and 100 µg ml⁻¹ of streptomycin (Gibco), 1X Glutamax (Gibco), and 15 mM HEPES (Gibco), 1% insulin-transferring-selenium (Sigma-Aldrich), 100 nM dexamethasone (Sigma-Aldrich), 50 mg/mL ascorbate-2-phosphate (Sigma-Aldrich), 10 ng/mL TGFb3 (Prepotech), and 10 ng/mL BMP6 (R&D Systems).

### Immunostaining

For fluorescence analysis, 10 mm thickness slices from the bioscaffolds were washed three times in PBS1X and permeabilized for 15 min in PBS 1X-0.1% TritonX-100 (PBT). Antigen retrieval was performed by adding 1 mg/mL Hyaluronidase (SIGMA, #H6254) diluted in PBS 1X and incubating 30 min at room temperature. After three washes 5 min each in PBS 1X, samples were dropped in Blocking solution (10% goat serum diluted in PBT) for 60 min at room temperature and then incubated overnight at 4° C. with mouse anti-human Collagen II (#II6B3, DSHB) diluted 1:250 in blocking solution. The day after, samples were washed three times for 10 min each and secondary antibody diluted 1:100 in blocking solution was added (anti-mouse IgG Alexa Fluor-647, #715-605-151, Jackson Immuno Research) and incubated for 2 h at room temperature. After three washes 5 min each in PBS 1X, the sections were washed three times 5 min each in PBS 1X, mounted with Fluoromount-G (Southern Biotech, Birmingham, AL, USA) onto glass slides. Pellet sections were imaged with a NikonA1R confocal microscope using a Nikon Plan VC 20x DIC N2 N.A. 0.75 objective lens and “Scan large image” from NIS-Elements software tool was used to image a larger field of view. Digital images were processed using NIS-Elements software (Nikon, Amsterdam, Netherlands) and Photoshop software (Adobe) was used to assemble the figure panels.

### RT-qPCR

Total RNA from bioscaffolds, were harvested at indicated time points using Tri Reagent (Ambion, Austin, TX, USA) according to the manufacturer’s protocol. DNA contamination were digested by DNAse I (Sigma). Reverse transcription (RT) was performed using Multiscribe reverse transcription kit (Thermo Scientific) following the manufacturer’s protocol. The relative amounts of COl2A1 and GAPDH RNAs were evaluated with TaqMan Gene expression assay (Applied Biosystems, Foster City, CA, USA) using the following probes: COL2A1 (Hs00264051_m1) and GAPDH (Hs02786624_g1) as housekeeping gene. qPCR was performed on a QuantStudio 6 Flex Real-Time PCR System (Thermo Fisher Scientific) and relative quantification was calculated with the 2E-ΔΔCT method.

### Mechanical Tests

The tests were performed at room temperature using a TA Electroforce 5500 mechanical loading device (TA Instruments, New Castle, USA) fitted with a calibrated 22 N load cell. The contact point between the two plates was recorded. Then, the sample was placed between two 4.2 cm diameter compression plates, in an unconfined setting. The displacement of the upper plate was controlled by a ramp function, at a rate of 0.01 mm/s, until a total displacement of 25% of the sample height. The contact area of the sample with the plate was measured by microscopy imaging before the test. Additionally, the point of inflexion of the load versus time curve showed the contact point between the sample surface and the compression plate to give the sample height. Load and displacement measurements were converted into stress (σ) and strain (ε) data using the sample surface area and height. The compressive modulus was then computed using stress data between 10 and 15% strain as follows: E_{c} = (σ₁₅-σ₁₀)/(ε₁₅ - ε₁₀).

### Example 2 - Harvesting

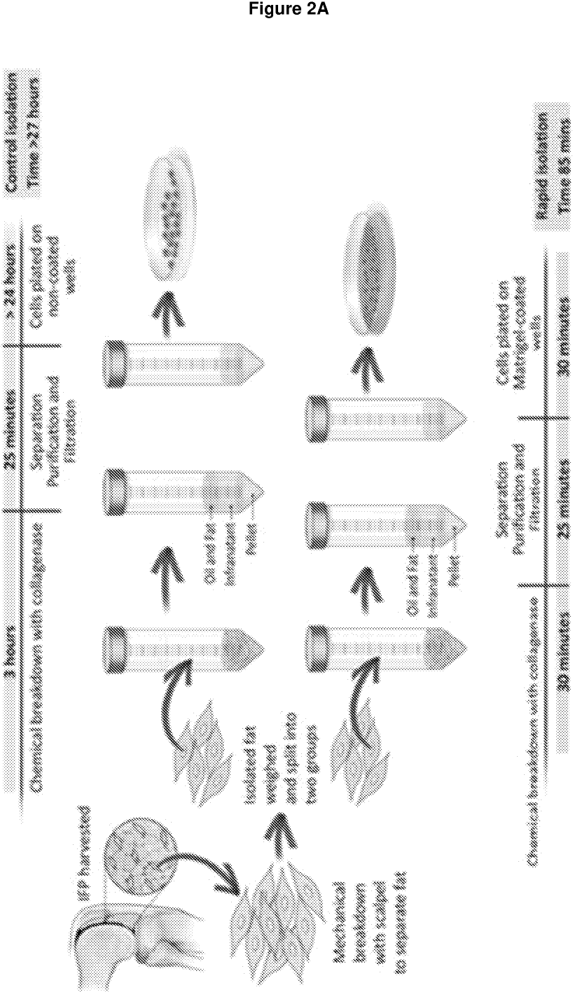

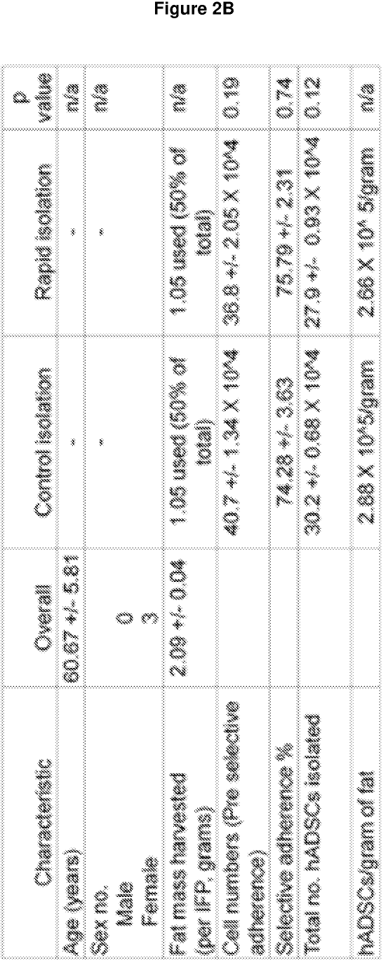

Human IFP was opportunistically harvested from three patients undergoing elective total knee arthroplasty. The tissues were weighed, and the number of cells isolated was evaluated at the end of the procedure. On average, IFPs comprises about 2 to 3 grams and about 8x10⁵ cells of which about 6x10⁵ cells are ADSCs, therefore there are about 3x10⁵ ADSCs per gram of fat tissue in the IFP (FIG. 2B).

### Example 3 - Enzymatic Digestion and Centrifugation

To speed up the stem cells isolation procedure, the inventors hypothesized that the duration of chemical breakdown and cell adherence could be reduced (FIG. 2A). Three IFPs from three different patients were isolated and each fat pad was weighed and divided equally into two. Cell isolation was performed using either rapid or control (standard) isolation procedures. Demographic characteristics of the three patients were all comparable (FIG. 2B). To test if the time required for chemical breakdown could be reduced, the inventors firstly evaluated the post isolation cell count and cell viability in both approaches to test if there was any reduction in cell yield or change in toxicity associated with using only 30 minutes of collagenase digestion (FIGS. 2B and 2C). The rapid isolation approach (30 minutes of collagenase digestion) yielded a cell count pre-selective adherence and cell viability comparable to the control isolation approach (3 hours of collagenase digestion) with no significant difference observed.

With this set of experiments, the inventors demonstrated that a standard protocol of stem cells isolation can be optimized to happen in only 85 minutes by means a reduced time in collagenase and adhesion on a surface substrate different from plastic. Those concepts are the preliminary findings that drove to the conceptualization of a Universal Polymer approach.

### Example 4 - Cells Adherence on Layer

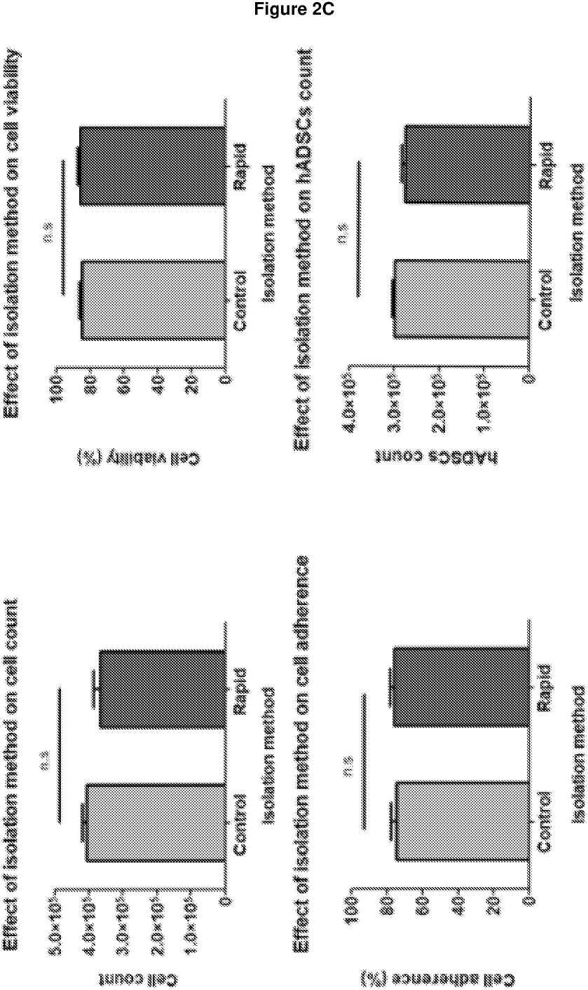

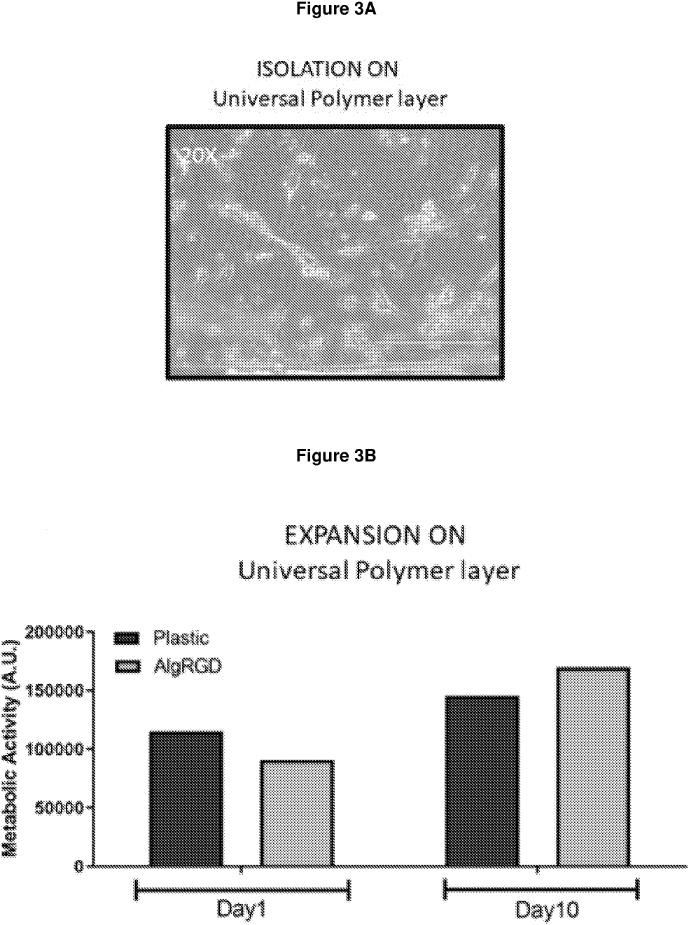

The adherence of the ADSCs isolated from IFP as described, was passed on a layer of a Universal Polymer (Alg-RGD1%). The cells were attached just only 30 minutes and monitored during time via microscopy imaging. A cell count was performed after 7 days showing that 300,000 cells were generated on an area of 9.6 cm² and a volume of 1 mL of Universal Polymer. The corresponding concentration obtained with the layer method was equal to 300,000 cell/mL (FIG. 3A). The cells display a polygonal shape typical of mesenchymal stem cells.

### Example 5 - Cells Expansion on Layer

The expansion of pre-isolated hADSCs was then tested on a layer of an example of Universal Polymer (Alg-RDG1%) using a metabolic activity assay. Despite the higher signal observed in the plastic control after 1 day from the adhesion, the fold changes in a Universal Polymer system was 1.86 compared to 1.26 in the plastic control (FIG. 3B). Those data demonstrated that hADSCs can expand on a Universal Polymer.

### Example 6 - Formulation of Bioink (Phase Change on Layer)

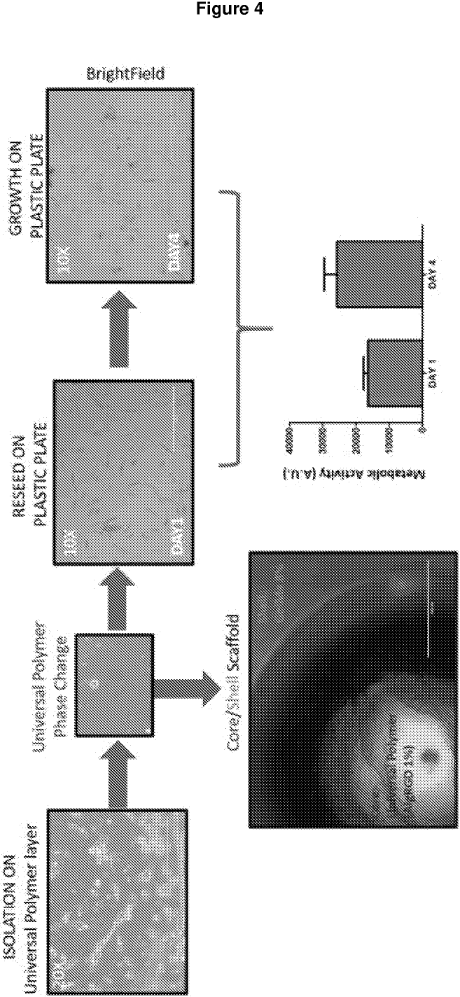

In order to test the survival of hADSCs isolated and expanded on a Universal Polymer, the inventors treated the Alg-RGD layer with 250 mM EDTA for 10 minutes. Once the phase change was completed and the Alginate liquified, half of the solution containing the cells was replated on a plastic surface to assess the cells survival through the phase change process. The second half was then used to generate a 3D bioscaffolds by means of co-axial extrusion. The liquefied Universal Polymer containing hADSCs constituted the Core compartment, while the Shell was constituted by GelMa 8% which was hardened via a photo-crosslinking reaction. The results of the metabolic assay showed that cells survived the phase change process and the metabolic activity increased by 1.56 fold after 4 days in culture (FIG. 4).

### Example 7 - Cells Adherence on 3D Particles

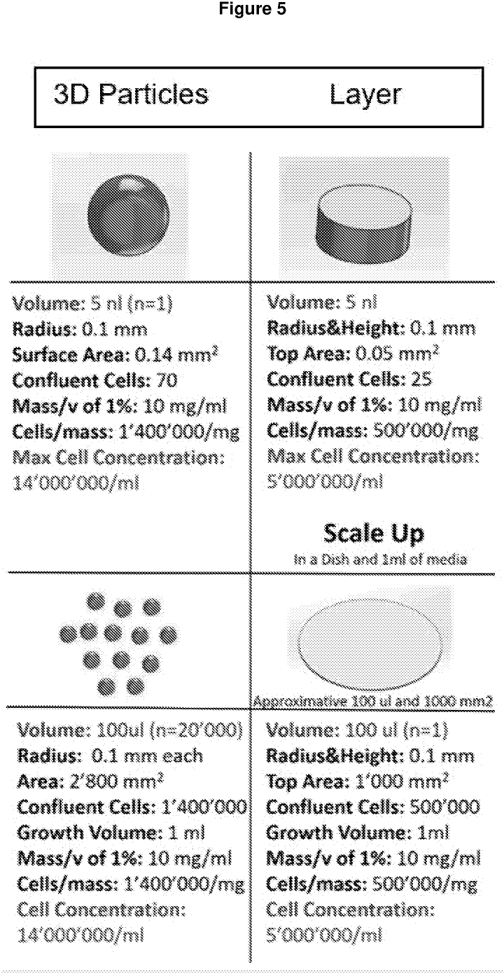

Despite the ability of hADSCs to adhere at the isolation and expand on a flat layer of Universal Polymer, the inventors estimated that in order to reach a higher concentration of cells in a smaller volume of polymer, able to repair a cartilage defect, they needed to improve the surface:area ratio, as shown in FIG. 5. Therefore, the inventors calculated that by using 3D particles they could achieve a cell concentration 3 times higher than the layer system. Moreover, the 3D particles, can be cultivated in a spinning bioreactor that works as expansion system and for cell banking at the same time. The bioreactor can significantly improve the expansion rate of the cells onto 3D particles due to the absence of any surface limiting constrain.

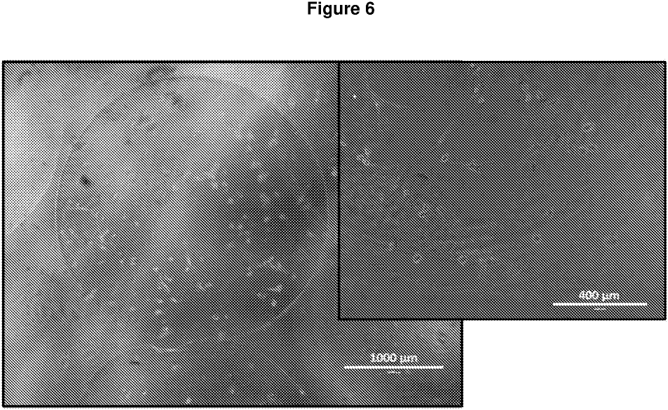

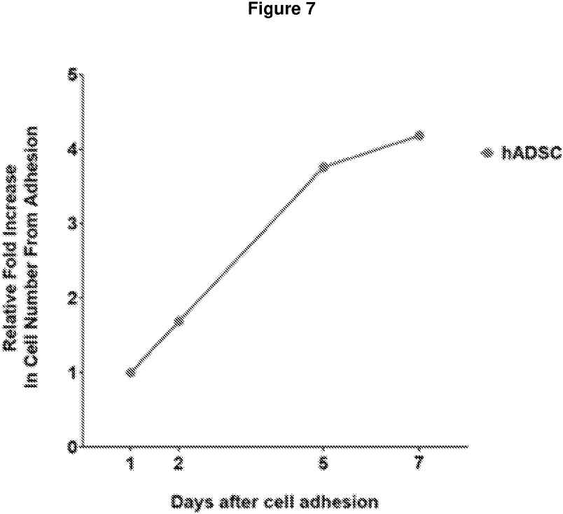

Alginate 3D particles (in the form a sphere) are produced by dropping a 1% (w/v) AlgRGD solution onto a bath of 18-180 mM CaCl2. Following their creation, cells are allowed to adhere to the 3D particles (FIG. 6) within a time frame of 1-4 hours.

### Example 8 - Cells Expansion on 3D Particles

The expansion of hADSC was tested on latex (“Cytodex”) 3D particles to assess the advantage of using a spinner flask Bioreactor system to cultivate stem cells. Expansion of hADSCs was evaluated by metabolic assay and showed a fold increase of 4.2 times over 7 days of culture.

### Example 9 - Formulation of Bioink (Phase Change on 3D Particles)

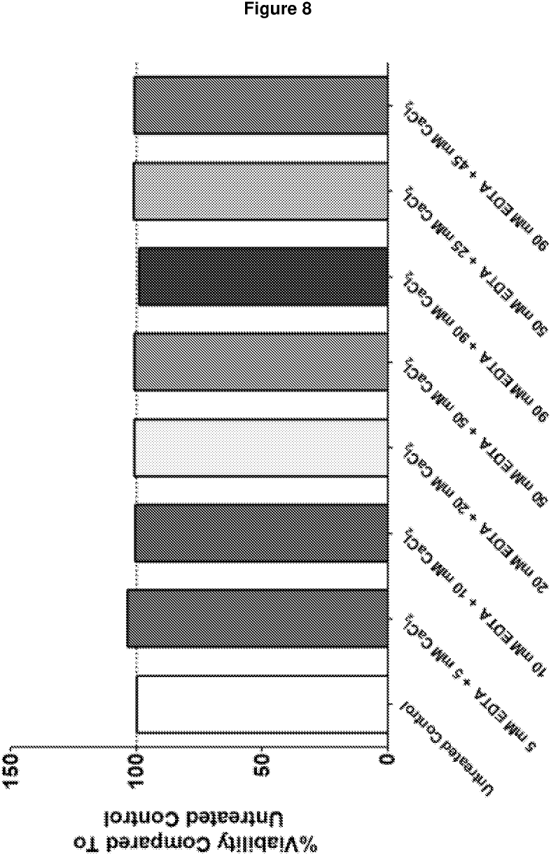

Once hADSCs have adhered to alginate spheres and expanded appropriately, the harvesting step can be undertaken through the use of EDTA chelation, as a means of reversing the calcium chloride-induced cross-linking of alginate spheres. The inventors identified a minimal EDTA treatment which does not affect cell viability when in presence of CaCl₂ (FIG. 8).

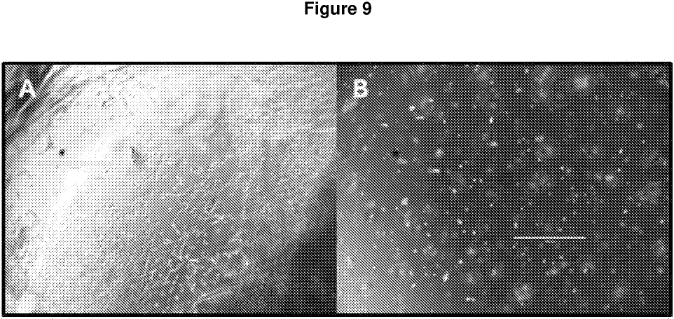

Thus, the inventors selected 90 mM EDTA as the most efficient non-toxic concentration of chelating agent which was able to revert the crosslinking of the 3D particles in only 10 minutes, thus generating the bioink (FIG. 9).

### Example 10 - Delivery

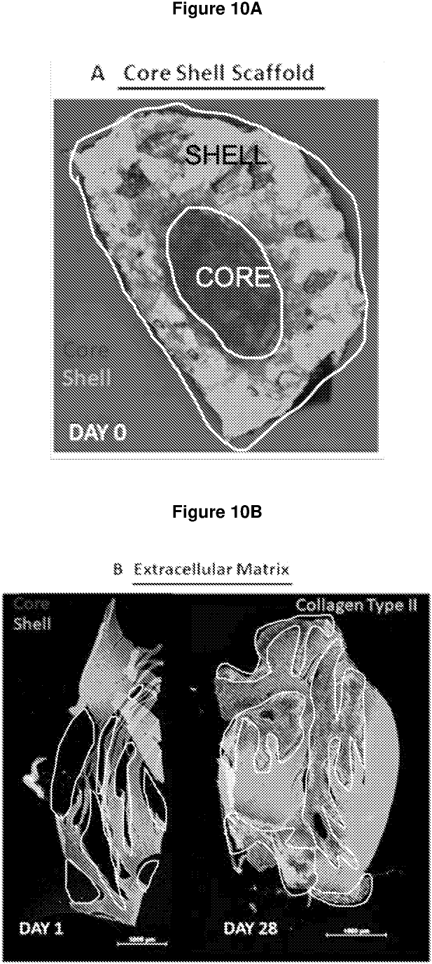

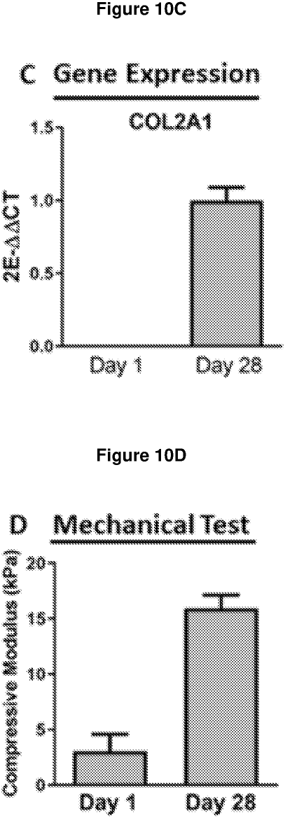

The generation of a Core/Shell Bioscaffold (FIG. 10A) constituted by a liquified core and a hardened shell allows for the delivery and the production of hyaline cartilaginous extracellular matrix. After 28 days of chondrogenic stimulation in vitro hADSCs were able to construct de novo cartilage tissue, which was composed by collagen type II (FIGS. 10B, C), the main marker of articular cartilage. Moreover, the bioscaffolds acquired an increased stiffness, which is mandatory to achieve bear loading capabilities (FIG. 10D). Similar results were also obtained with the same cell source, using a homogeneous scaffold constituted by a single photocrosslinkable material component based on Gelatine (GelMa) both in vitro and in an in vivo rabbit model.

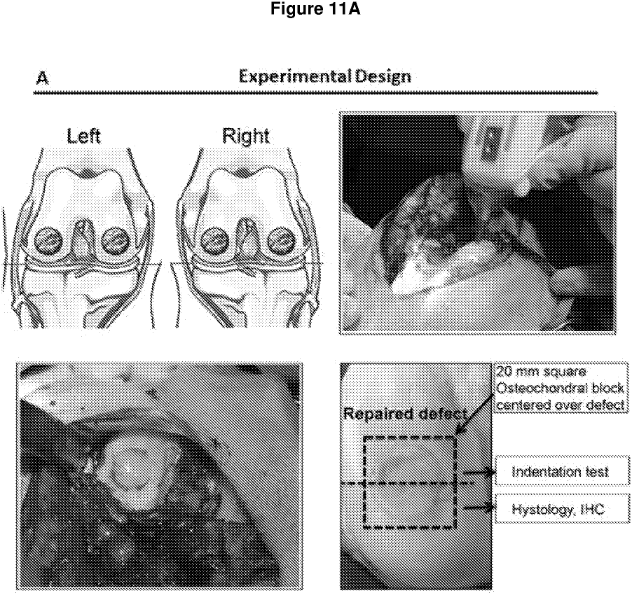

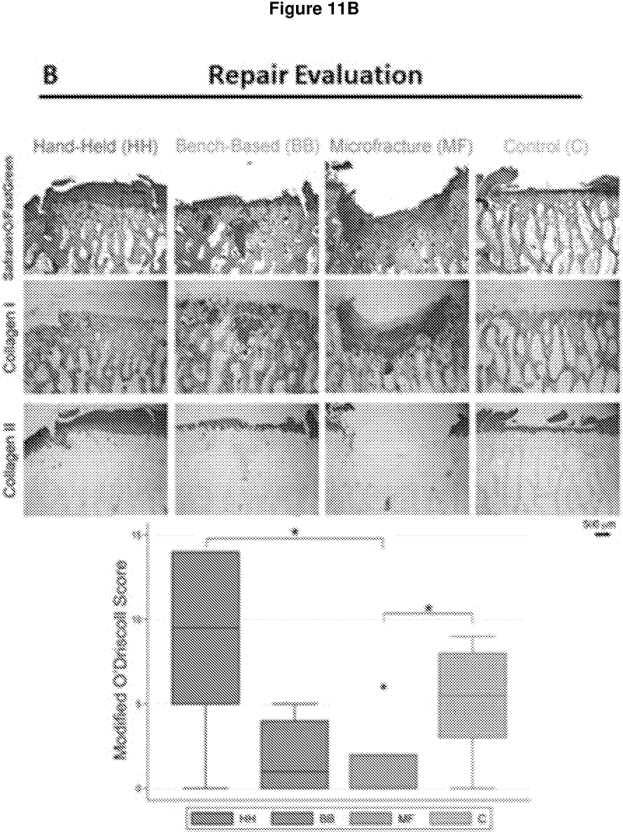

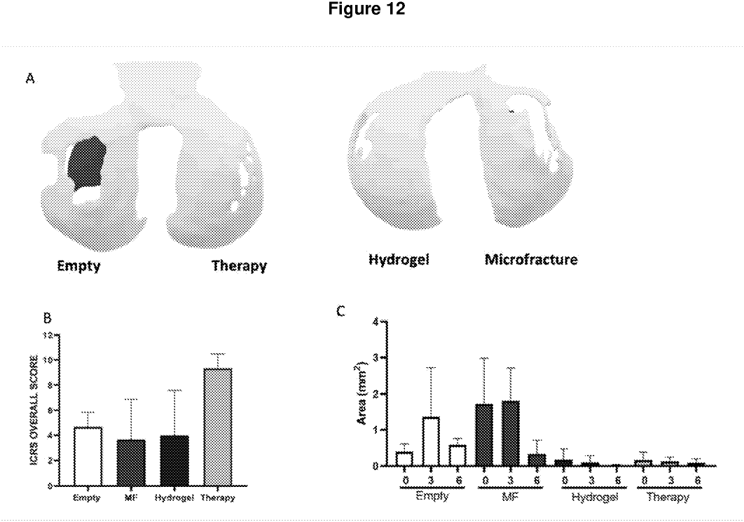

To confirm the chondrogenic potential of the delivery strategy the inventors performed a pilot in vivo animal study on a full chondral defect model in sheep (FIG. 11A; (For technical Details, see. Di Bella et al. J Tissue Eng Regen Med. 2018. 12(3):611-621). Results showed a significant level of cartilage repair after 6 weeks when the defects were treated with the in situ generated Core/Shell bioscaffold, respect to the poor repair observed in the microfracture gold standard treatment (FIG. 11B). In a larger 6 month study the inventors tested the chondrogenic potential of hADSCs delivered with an improved coaxial extrusion device (Biopen). The Bioscaffolds were constituted by ALG-RGD 1% in the core and GelMa 10% in the Shell, matching the same composition of the Universal Polymer tested in vitro (FIG. 12).

It will be understood that the invention disclosed and defined in this specification extends to all alternative combinations of two or more of the individual features mentioned or evident from the text or drawings. All of these different combinations constitute various alternative aspects of the invention.

1. A method for forming a cell composition from a tissue sample, the method comprising:
- providing a tissue sample comprising cells;
- contacting the sample with a polymer in binding conditions, said binding conditions being conditions that enable binding of cells in the sample to the polymer, so that said cells are bound to the polymer;
- culturing the cells bound to the polymer under conditions and for a time that allows the cell number to increase;
- providing conditions to induce a phase change of the polymer, wherein the cells remain bound to the phased changed polymer;
thereby forming a cell composition from a tissue sample.

2. The method of claim 1, further comprising a step of isolating the cells from the extracellular matrix in the tissue sample.

3. The method of claim 2, wherein isolating the cells from the extracellular matrix is performed by mechanical disruption.

4. The method of claim 2, wherein isolating the cells from the extracellular matrix is performed by enzymatic digestion.

5. The method of any one of claims 2 to 4, wherein isolating the cells from the extracellular matrix is performed by mechanical disruption and enzymatic digestion.

6. The method of any one of claims 2 to 5, wherein isolating the cells separates the cells from any fat lobules in the sample.

7. The method of claim 4 or 5, wherein the enzymatic digestion is performed with collagenase.

8. The method of claim 7, wherein the collagenase is used at a specific activity of 2 U/ml for a period of 30 minutes or less.

9. The method of any one of claims 2 to 8, further comprising the step of separating the isolated cells from substantially all the fat and/or liquid present in the sample.

10. The method of claim 9, wherein separating the isolated cells may be performed by centrifugation.

11. The method of claim 10, wherein the centrifugation is performed at about 2000 g for about 5 minutes to form a cell pellet.

12. The method of claim 11, wherein the cell pellet is resuspended in a buffer for lysis of red blood cells.

13. The method of claim 12, further comprising filtering the cells in the lysis buffer to separate debris from viable cells and further centrifugation for about 5 minutes at about 400 g to obtain a further cell pellet.

14. The method of claim 1, wherein the polymer is capable of attaching to a solid phase of a particle, vessel or device, or capable of forming a particle, in said binding conditions.

15. The method of any one of claims 1 to 14, wherein the polymer is capable of binding to cells in said binding conditions that are human adipose derived stem cells (ADSCs) or hADSC precursor cells, or to cells that are derived from hADSC that are chondrogenic or that have chondrogenic potential.

16. The method of any one of claims 1 to 15, wherein the polymer is not capable of binding to fibroblasts in said binding conditions.

17. The method of any one of the claims 1 to 16, wherein the polymer comprises a peptide or protein.

18. The method of claim 17, wherein the peptide or protein binds to an extracellular matrix adhesion receptor.

19. The method of claim 18, wherein the extracellular matrix adhesion receptor is an integrin receptor.

20. The method of claim 17, wherein the peptide or protein comprises an integrin binding motif.

21. The method of claim 20, wherein the integrin binding motif is RGD.

22. The method of claim 17, wherein the peptide comprises or consists of GGGGRGDSP, GRGDSP or GRGDS, or an amino acid sequence with 1 or 2 amino acid insertions, deletions, substitutions (preferably conservative substitutions) or a combination thereof.

23. The method of any one of claims 1 to 22, wherein the polymer is capable of reversible liquid-solid phase change.

24. The method of any one of claims 1 to 23, wherein the polymer is capable of a liquid to solid phase change caused by an ionic crosslinking agent.

25. The method of claim 24, wherein the ionic crosslinking agent is a divalent cation.

26. The method of claim 25, wherein divalent cation is Ca²⁺.

27. The method of any one of claims 1 to 26, wherein the polymer is capable of a solid to liquid phase change caused by the chelation of an ionic crosslinking agent.

28. The method of claim 27, wherein the chelation occurs by presence of a chelating agent capable of chelating an ionic crosslinking agent.

29. The method of claim 26, wherein the chelating agent is EDTA.

30. The method of any one of the claims 1 to 29, wherein the polymer comprises gelatin or a derivative thereof.

31. The method of claim 30, wherein the gelatin polymer is Gelatin methacryloyl (GeIMA).

32. The method of any one of claims 1 to 29, wherein the polymer comprises alginate or derivative thereof.

33. The method according to claim 32, wherein the polymer comprises alginate-RGD.

34. The method according to any one of claims 1 to 33, wherein the polymer is capable of photo-crosslinking.

35. The method according to claim 34, wherein the photo-crosslinking is mediated by a reactive functionality capable of photo-crosslinking present in the polymer.

36. The method according to claim 35, wherein the reactive functionality is a methacryloyl group.

37. The method of any one of claims 1 to 36, wherein the polymer comprises alginate, an RGD motif and a methacryloyl group.

38. The method of any one of claims 1 to 37, wherein the tissue sample comprises a first cell type and a second cell type and wherein the binding conditions enable the binding of the first cell type to the polymer and wherein the binding conditions do not enable binding of the second cell type to the polymer.

39. The method of claim 38, wherein separation of the polymer from the tissue sample forms a cell composition consisting of cells of the first cell type, and forms a waste stream comprising cells of the second cell type.

40. The method of claims 38 or 39, wherein the first cell type is a hADSC or chondrogenic cell and the second cell type is a fibroblast.

41. The method of any one of claims 1 to 40, wherein the tissue sample is obtained from the infrapatellar fat pad.

42. The method of claim 41, wherein the fat pad has a weight of about 2 to 3 g.

43. The method of any one of claims 1 to 42, wherein the polymer is in the form of a 3D particle.

44. The method of claim 43, wherein the sample is contacted with the 3D particle in a bioreactor at a cell:particle ratio of about 10 cells to every particle.

45. The method of any one of claims 1 to 44, wherein the step of culturing the cells allows at least 2 cycles of cell divisions.

46. The method of any one of claims 1 to 44, wherein the step of culturing the cells is for a period of at least 5, at least 6 or at least 7 days.

47. The method of any one of claims 1 to 44, wherein the step of culturing the cells results in an increase of about 3-4 times the original cell number.

48. The method of any one of claims 1 to 44, wherein the step of culturing the cells results in about 5 million cells.

49. The method of any one of claims 1 to 48, wherein when the tissue sample contains stem cells, preferably ADSCs, the method further comprises the step of priming of those stem cells to differentiate into a cell type of interest, for example, priming of ADSCs to form chondrocytes.

50. The method of claim 49, wherein the priming step occurs at the same time or subsequent to culturing the cells that allows an increase in cell number.

51. The method of any one of claims 1 to 50, wherein the conditions to induce a phase change of the polymer is heating.

52. The method of any one of claims 1 to 51, wherein the conditions to induce a phase change of the polymer is application of a chelating agent.

53. The method of claim 52, wherein the chelating agent is EDTA.

54. The method of any one of claims 51 to 53, wherein the phase change increases the flowability of the polymer enabling the cell composition to be administered to an individual at room temperature by injection, extrusion or 3D printing.

55. The method of claim 51 wherein the heating step comprises heating the cell composition to a temperate that does not affect the viability of the cells in the cell composition.

56. The method of anyone of claims 1 to 55, wherein the polymer has a melting temperature of about 25 to 30° C.

57. A method for treating an individual comprising:
- forming a cell composition according to any one of the preceding claims, or being provided with a cell composition formed according to any one of the preceding claims ;
- administering the cell composition to the individual,
thereby treating the individual.

58. The method of claim 57, wherein the cell composition is formed from a tissue sample obtained from the individual.

59. The method of claim 58, wherein the cell composition is formed from a tissue sample obtained from an infrapatellar fat pad of the individual.

60. The method of any one of claims 57 to 59, wherein the cell composition is administered by injection, extrusion or 3D printing.

61. The method of any one of claims 57 to 60, wherein the cell composition is administered with a further polymeric composition so that the further polymeric composition coats the cell composition as the cell composition is administered to the individual.

62. The method of claim 61, wherein the further polymeric composition is photocrosslinkable.

63. The method of any one of claims 57 to 62, wherein the individual has a condition of an articular surface requiring repair or restoration.

64. The method according to any one of claims 57 to 63, wherein the cell composition is administered to an articular surface requiring repair or restoration.

65. The method according to any one of claims 57 to 64, wherein the cell composition is administered arthroscopically, preferably with ultrasound or imaging guidance.

66. The method according to any one of claims 57 to 64, wherein the cell composition is administered upon open surgery.

67. The method according to any one of claims 57 to 66, wherein the delivered cell composition is hardened by the activation of a photoinitiator.

68. The method of claim 67, wherein the photoinitiator is activated with visible light.

69. The method of claim 68, wherein the photoinitiator is LAP.

70. The method of claim 68 or claim 69, wherein a 405 nm light source at 20 mW/cm² is applied for 1 minute.

71. A cell composition obtained by a method of any one of claims 1 to 56.

72. A cell composition obtainable by a method of any one of claims 1 to 56.

73. Use of a cell composition of claim 71 in the manufacture of a medicament for treatment of a condition requiring re-implantation of cells for said treatment.

74. A cell composition of claim 71 for use in the treatment of a condition requiring implantation of cells for said treatment.

75. A cell composition of claim 71 when used for treatment of a condition requiring implantation of cells for said treatment.

76. A kit for use, or when used, in a method of any one of claims 1 to 70, the kit comprising a polymer as defined in any one of claims 1 to 70.

77. The kit of claim 76, further comprising written instructions to perform a method of any one of claims 1 to 70.

78. Use of a cell composition of claim 72 in the manufacture of a medicament for treatment of a condition requiring re-implantation of cells for said treatment.
